(19) United States
(12) Patent Application Publication
(10) Pub. No.: US 2024/0257920 A1
(43) Pub. Date: Aug. 1, 2024

(54) Systems and Methods for Multipart Molecule Fragment Determination and Scoring

(71) Applicant: DH Technologies Development Pte. Ltd., Singapore (SG)

(72) Inventors: Lyle BURTON, Concord (CA); Carolyn CARKNER, Woodbridge (CA); Eva DUCHOSLAV, Toronto (CA)

(21) Appl. No.: 18/565,716

(22) PCT Filed: May 27, 2022
(86) PCT No.: PCT/IB2022/054984

§ 371 Date: Nov. 30, 2023

Related U.S. Application Data
(60) Provisional application No. 63/195,148, filed on May 31, 2021.

(51) Int. Cl. G16C 20/20; G16C 20/70; H01J 49/00

(52) U.S. Cl. CPC G16C 20/20; G16C 20/70; H01J 49/0045

(57) ABSTRACT

In one aspect, a computer implemented method for determining expected cleavage products of a macromolecule includes defining at least one residue of the macromolecule as having a core and at least one linker, where said at least one linker is defined as a sequence of two or more structural units that are coupled to one another via one or more chemical bonds. A digital data processor can be utilized to determine one or more expected bond cleavages, if any, between the structural units of said at least one linker and between adjacent residues when the macromolecule undergoes cleavage, e.g., in response to application of energy thereto, so as to predict expected cleavage products of the macromolecule.

### BACKGROUND

The present teachings are generally directed to methods and systems for determining expected cleavage products of a variety of molecules, such as oligonucleotides, peptides, among others.

Mass spectrometry (MS) is an analytical technique for determining the structure of test chemical substances with both qualitative and quantitative applications. MS can be useful for identifying unknown compounds, identifying atomic elements in a molecule, determining the structure of a compound by observing its fragmentation, and quantifying the amount of a particular chemical compound in a mixed sample. Mass spectrometers detect chemical entities as ions such that a conversion of the analytes to charged ions must occur during the sampling process.

MS spectrometry is increasingly employed for determination of structures of peptides and oligonucleotides. For example, the use of synthetic oligonucleotides for therapeutic applications and research applications is rapidly advancing. The confirmation of the residue sequences of such synthetic oligonucleotides is essential in ensuring that the oligonucleotides employed in these applications do in fact possess the expected residue sequence.

### SUMMARY

In one aspect, a computer implemented method for determining expected cleavage products of a macromolecule having a plurality of residues bonded to one another is disclosed, which includes defining at least one of those residues as having a core and at least one linker, where said at least one linker is defined as an assembly of two or more structural units that are coupled to one another via one or more chemical bonds. The macromolecule can be represented as a sequence of the residues such that each residue is bonded to an adjacent residue via at least one chemical bond. A digital data processor can be utilized to determine one or more expected bond cleavages, if any, between the structural units of said at least one linker and between adjacent residues when the macromolecule undergoes cleavage so as to determine expected cleavage products of the macromolecule. By way of example, the cleavage of the macromolecule can occur as a result of application of energy thereto. The energy applied to the macromolecule can be generated via a variety of different sources and mechanisms. By way of example, the energy can be generated via a chemical reaction, particle impact, or via a radiation source (e.g., an ultraviolet radiation source), among others.

In some embodiments, at least one of the linker structural units can include a plurality of atoms that are bonded to one another via one or more chemical bonds. The digital data processor can be configured to determine expected bond cleavages. Typically, bond cleavages between the structural units of the residues and between adjacent residues are considered for determining the expected cleavage products.

In some embodiments, the core of said at least one of said residues can be represented as an assembly of at least two structural units that form one or more chemical bonds with one another. The digital data processor can further be configured to determine one or more expected cleavages of the chemical bonds between the core structural units, if any, and to use the expected core cleavages, in addition to the expected linker bond cleavages, if any, to determine the expected cleavage products of the macromolecule.

The present teachings can be employed to determine the expected cleavage products of a variety of different types of macromolecules. Some examples of such macromolecules include, without limitation, oligonucleotides, peptides, among others.

In some embodiments, at least one residue of an oligonucleotide can be defined as having a core and a linker, where the core is represented by a base and a sugar structural unit that are chemically bonded to one another. The base and the sugar structural units can be in the form of non-standard base and/or sugar moieties. Further, the linker structural units of such a residue can include a phosphorus-containing group, a 5′ linker and a 3′ linker. Each of the 5′ linker and the 3′ linker can include one or more atoms. Further, in some embodiments, any of the 5′ linker and the 3′ may be missing.

In some embodiments, the residue can include any of an amino, a hydroxy-amino and a carbonyl group. For example, such a residue can be one of the residues of a peptide, as that term is used herein. In some such embodiments, at least one of the structural units of the linker can include a silicon atom. For example, at least one of the structural units of the linker can include Si(OH)₂.

In some embodiments, the energy for causing the cleavage of the macromolecule can be generated via any of hydrolysis, electron capture dissociation, electron impact dissociation, collision-induced dissociation, depurination and depyrimidation.

In some embodiments, the method can further include assigning a unique symbol to the defined residue so as to generate a symbolic representation of that residue.

Further, in some embodiments, experimentally-observed cleavage products of a target macromolecule can be compared with the expected cleavage products to determine a degree of correspondence between the molecular structure of the target macromolecule and that of the putative macromolecule. In some embodiments, the definition of the macromolecule (e.g., in the form of symbols for its residues) can be stored in a database, e.g., a relational database.

In a related aspect, a computer implemented method for determining expected cleavage products of an oligonucleotide is disclosed, which includes defining the oligonucleotide as a sequence of a plurality of residues, where each residue is represented using five distinct structural units comprising a base, a 5′ linker, a sugar, a 3′ linker and a linking unit for linking adjacent residues via any of said 5′ and 3′ linkers. A digital data processor is used to determine a plurality of expected cleavage products when the oligonucleotide undergoes cleavage. By way of example, the oligonucleotide can undergo cleavage in response to application of energy thereto. As noted above, the energy applied to the oligonucleotide can be generated using a variety of different energy sources and mechanisms. For example, as discussed above, the energy can be generated via any of a chemical reaction, particle impact, and a source of radiation (e.g., a source of ultraviolet radiation, e.g., in a range of about 200 nm to about 300 nm). In some embodiments, the energy can be generated via any of hydrolysis, electron capture dissociation, electron impact dissociation, collision-induced dissociation, depurination and depyrimidation.

In a related aspect, a system for determining expected cleavage products of a putative macromolecule having a plurality of residues bonded to one another is disclosed, which comprises at least one microprocessor, a user interface operating under control of said microprocessor, said user interface comprising at least one user interface element configured to allow a user to define at least one residue of said macromolecule as a plurality of structural units chemically bonded to one another, said at least one user interface element further allowing the user to define the macromolecule as a sequence of chemically-bonded residues comprising said at least one residue and one or more additional residues, and a predictive module operating under control of the microprocessor and being in communication with said user interface for receiving information regarding the residues. The predictive module can be configured to determine one or more expected bond cleavages of the macromolecule when the macromolecule undergoes cleavage so as to determine expected cleavage products of the macromolecule.

In some embodiments, the predictive module is configured to determine the expected cleavage products of the macromolecule by determining one or more chemical bonds of that macromolecule that are expected to break when the macromolecule is subjected to application of an energy generated by an energy source, and identifying cleavage products generated in response to breakage of those bonds, individually and in combination with other bonds.

In some aspects, the structural units of residues of a macromolecule are selected such that the breakage of chemical bonds between such structural units and between the adjacent residues will generate the most significant cleavage products of the macromolecule.

For example, in some embodiments, it is assumed that the most characteristic bond cleavages occur between the structural units of each residue of a defined macromolecule (e.g., an oligonucleotide) and between its adjacent residues. In some cases, it is assumed that all of the bonds between the structural units and/or between the adjacent residues are susceptible to cleavage. In some embodiments, such an approach advantageously allows the determination of the most significant cleavage products. In other embodiments, one or more bonds within a defined structural unit may be also considered as being susceptible to cleavage.

The user interface can be configured to allow a user to assign a unique symbol to said at least one residue so as to provide a symbolic representation thereof. By way of example, the symbolic representation of the residue can include a plurality of alphanumeric characters. In some embodiments, such a symbolic representation of the residue can be stored in a database such that it can be retrieved for use in defining a macromolecule. For example, in some embodiments, a macromolecule can be defined by specifying its residues, where one or more residues can be user-defined residues retrieved from a database in which the definition of the residue was previously stored, and one or more residues can be standard residues (e.g., standard nucleotides and/or amino acids). Other definitions of a macromolecule is also possible, e.g., a definition in which all of the residues are custom user-defined residues.

In some embodiments, the user interface can be configured to allow a user to indicate that at least one of the structural units of a user-defined residue is missing. In other words, in some embodiments, the user interface can provide the option of identifying a number of structural units (e.g., 5 structural units) for a user-defined residue and further allow a user to leave the definition of one or more of those structural units as blank (i.e., to indicate that those structural units are missing in that residue). This allows considerable flexibility in defining one or more residues of a macromolecule.

Further understanding of various aspects of the present teachings can be obtained by reference to the following detailed description in connection with the associated drawings, which are described briefly below.

### BRIEF DESCRIPTION OF THE DRAWINGS

FIG. 1A shows the standard ion series that can be observed during MS/MS fragmentation of a standard oligonucleotide,

FIG. 1B is a flow chart depicting various steps in a computer-implemented method according to an embodiment for determining expected cleavage products of a macromolecule,

FIG. 2 is a flow chart depicting various steps according to an embodiment of a method according to the present teachings for determining the expected cleavage products of a macromolecule and optionally comparing the expected cleavage products with experimentally-observed cleavage products of a target macromolecule,

FIG. 3 is an example of a user-defined oligonucleotide residue and a user interface element that allows the user to specify various structural units of the residue,

FIG. 4 is an example of a user interface element that allows a user to specify one or more terminal moieties of an oligonucleotide,

FIG. 5 schematically depicts that a user-defined residue can be stored in a database,

FIG. 6 schematically depicts a user interface element that allows a user to define an oligonucleotide via identification of the sequence of its residues,

FIG. 7 presents the ‘a’ and ‘b’ and ‘c’ masses of the expected cleavage products of the oligonucleotide defined via the interface element shown in FIG. 6, when the oligonucleotide is subjected to MS/MS fragmentation,

FIG. 8 presents an experimentally-observed MS/MS spectrum of a five-residue oligonucleotide,

FIG. 9 is a table listing mass-to-charge ratios for some of the expected fragments of the oligonucleotide having the MS/MS spectrum depicted in FIG. 8,

FIGS. 10A and 10B show examples of peptides having custom residues defined in accordance with embodiments of the present teachings,

FIG. 11 schematically depicts an example of an embodiment of a system according to the present teachings for determining expected cleavage products of a macromolecule,

FIG. 12 is an example of a mass spectrometry system in which a predictive system according to an embodiment is incorporated, and

FIG. 13 is an example of hardware implementation of a system according to embodiments of the present teachings.

### DETAILED DESCRIPTION

It will be appreciated that for clarity, the following discussion will explicate various aspects of embodiments of the present disclosure, while omitting certain specific details wherever convenient or appropriate to do so. For example, discussion of like or analogous features in alternative embodiments may be somewhat abbreviated. Well-known ideas or concepts may also for brevity not be discussed an any great detail. One of ordinary skill will recognize that some embodiments of the present disclosure may not require certain of the specifically described details in every implementation, which are set forth herein only to provide a thorough understanding of the embodiments. Similarly it will be apparent that the described embodiments may be susceptible to alteration or variation according to common general knowledge without departing from the scope of the disclosure. The following detailed description of embodiments is not to be regarded as limiting the scope of the applicant's teachings in any manner.

As used herein, the terms “about” and “substantially equal” refer to variations in a numerical quantity that can occur, for example, through measuring or handling procedures in the real world; through inadvertent error in these procedures; through differences in the manufacture, source, or purity of compositions or reagents; and the like. Typically, the terms “about” and “substantially” as used herein means 10% greater or lesser than the value or range of values stated or the complete condition or state. For instance, a concentration value of about 30% or substantially equal to 30% can mean a concentration between 27% and 33%. The terms also refer to variations that would be recognized by one skilled in the art as being equivalent so long as such variations do not encompass known values practiced by the prior art.

As used herein the term “and/or” includes any and all combinations of one or more of the associated listed items and may be abbreviated as “/”.

The present disclosure relates generally to methods and systems for determining an expected cleavage pattern of a macromolecule when subjected to a cleavage process, e.g., in response to application of energy thereto. As noted above, the energy causing the cleavage may be supplied via a variety of different mechanisms and sources. Without limitation, in some embodiments, the cleavage of one or more chemical bonds of a macromolecule may occur when the macromolecule undergoes a fragmentation process during an MS/MS analysis.

As discussed in more detail below, in some embodiments, the experimentally-observed cleavage products of a target macromolecule (e.g., a target oligonucleotide) can be compared with the expected cleavage products of a putative macromolecule (e.g., an oligonucleotide) to determine a degree of correspondence between the molecular structure of the target macromolecule and that of the putative macromolecule. By way of example, such comparison between the molecular structure of a target macromolecule and that of a putative macromolecule can be used in confirming the molecular structure of a synthetic macromolecule that is expected to have a molecular structure corresponding to that of a putative macromolecule.

Though not limited to mass spectrometry or oligonucleotides, in one application of the methods and systems according to various embodiments, a plurality of experimentally-observed fragments of a target oligonucleotide can be compared with a plurality of expected fragments of a putative oligonucleotide (including a putative oligonucleotide having one or more non-standard user-defined residues) to determine a degree of correspondence between the molecular structure of the target oligonucleotide and that of the putative oligonucleotide.

There are a number of ways of comparing an experimental MS/MS spectrum to a list of expected masses. In some embodiments, initially, a mass tolerance can be defined. By way of example, such a mass tolerance can be based on the mass measurement accuracy that a mass spectrometer used to generate mass peaks corresponding to the experimentally-observed cleavage products is able to achieve. For example, such a mass measurement accuracy can be about 0.5 Da for a low-resolution quadrupole mass spectrometer or about 0.05 Da or lower for a higher-resolution time-of-flight (TOF) mass spectrometer.

Further, in some embodiments, an intensity threshold, e.g., in counts/second, can be defined such that any experimentally-observed mass peaks having an intensity below that threshold will be ignored as being most likely just “noise” and not a relevant mass signal.

The number of experimentally-observed mass peaks (those above a predefined threshold) which match (within the mass error tolerance) the respective expected mass peaks can then be calculated. Further, the total number of the experimentally-observed peaks that do not match (within the mass error tolerance) any of the predicted mass peak is also calculated.

It can be useful to have a measure of correspondence between the molecular structure of a target macromolecule and that of a putative macromolecule. By way of example, such a score can be defined as a fraction of all matching mass peaks relative to all experimentally-observed peaks as a measure of the degree of correspondence between the molecular structure of the target macromolecule (e.g., an oligonucleotide) and a putative macromolecule (e.g., a putative oligonucleotide).

The above score ranges from 0 to 1. In particular, if each of the experimentally-observed mass peaks matches an expected mass peak, i.e., there are no unmatched peaks, then the score is 1 and if there is no mass peak that would match an expected mass peak, the score is 0.

The score can be defined differently than the one described above. In fact, a variety of score definitions can be used for assessing the degree of correspondence between the molecular structure of a target macromolecule (e.g., an oligonucleotide or peptide) with that of a putative macromolecule (e.g., a putative oligonucleotide or a putative peptide).

The use of MS/MS spectrometry in which the fragments of a precursor ion are generated and analyzed is one of the most prevalent mass spectrometric techniques for the elucidation of the structures of peptides and oligonucleotides. The use of such a technique requires the determination of the expected MS/MS fragments (or at least the most prominent/significant MS/MS fragments) of an oligonucleotide or a peptide and comparing the expected fragments with the experimentally-observed fragments.

The determination of the expected MS/MS fragments of oligonucleotides can be more challenging than the determination of the expected MS/MS fragments of standard peptides. The determination of the most relevant MS/MS fragments of a standard peptide is straightforward since peptides undergo fragmentation to generate the well-known a, b, y, etc. ion series. Even when the residues of a peptide are modified, such modifications are typically made to side-chains of the peptide and not to its backbone. Thus, the fragment ion series remain unchanged (other than being shifted by the mass of the modification). It is, however, noted that the determination of the expected cleavage products of non-standard peptides can be more complicated.

In contrast to standard peptides, the inventors have recognized that oligonucleotides (e.g., DNA or RNA) may undergo modifications at a base side-chain, at a sugar portion of the backbone, the phosphate and/or to the 5′ and/or 3′ groups, which link neighboring nucleotides of the oligonucleotide. As such, an accurate determination of the possible MS/MS fragments of an oligonucleotide requires information about not only the position(s) that are modified but also the modifying entity (or entities).

The inventors have further realized that although an oligonucleotide residue may be defined as base/sugar/linker combinations, such an approach does not separate the sugar and the linker moieties with a sufficient granularity to allow an accurate determination of all expected cleavage products when the oligonucleotide undergoes cleavage.

As discussed in more detail below, embodiments of the present disclosure regarding methods and systems for identifying/confirming the nucleotide sequence of a target oligonucleotide are at least in part based on the recognition that defining an oligonucleotide as having five categories of structural units including a base moiety, a 5′ linker, a sugar moiety, a 3′ linker and a phosphate moiety can significantly increase the accuracy for the determination of MS/MS fragments that are expected to be generated during tandem mass spectrometric analysis of the oligonucleotide. This can in turn improve the accuracy in confirming that a target oligonucleotide (e.g., a synthetic oligonucleotide) does in fact contain the desired (expected) sequence of residues.

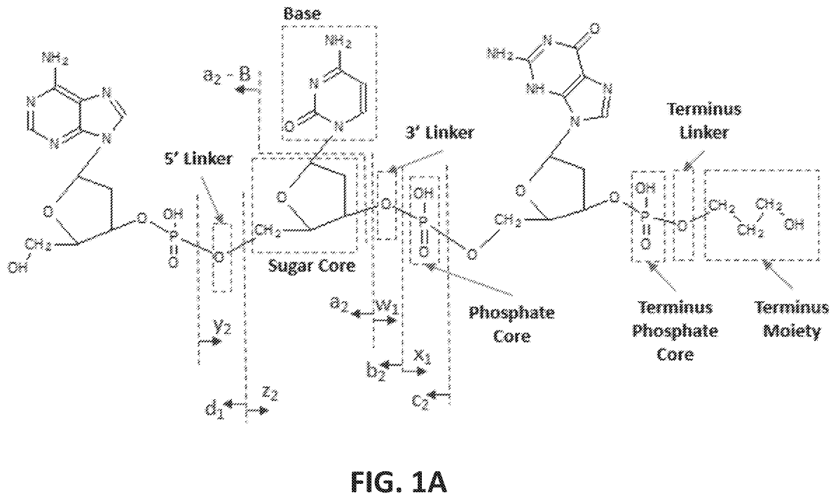

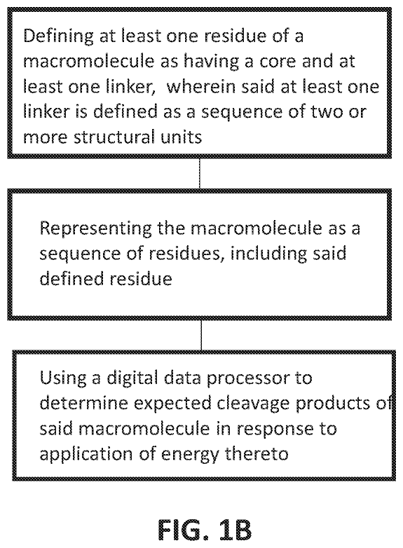

By way of illustration, FIG. 1A shows the standard ion series that can be observed during MS/MS fragmentation of a standard oligonucleotide. By representing each residue using five independent structural units, namely, a base moiety, a sugar moiety, a 5′ linker, a 3′ linker and a phosphate moiety, one can account for most significant (and in some cases all) of the generated cleavage products. Although the present teachings can be used to determine the expected cleavage products of standard oligonucleotides, the present teachings can be applied more broadly to a variety of macromolecules, including those that include custom user-defined residues, as discussed in more detail below. In embodiments, the present teachings provide computer-implemented methods and systems that allow defining a macromolecule via identification of a plurality of its residues, where each residue can be defined as having a core and a linker for coupling that residue to an adjacent residue. In particular, in some embodiments, the systems and methods according to the present teachings allow a user to define a core and/or a linker of at least one residue. In other words, the present teachings allow user-defined custom definition of one or more residues of a macromolecule.

The methods and systems according to the present teachings can further generate information regarding expected intra- and inter-residue bond cleavages when the macromolecule undergoes cleavage (e.g., in response to being subjected to a chemical reaction) and can utilize that information to determine expected cleavage products.

In some embodiments, a plurality of experimentally-observed cleavage products of a target macromolecule can be compared with a plurality of expected cleavage products of a putative macromolecule to determine a degree of correspondence between the molecular structure of the target macromolecule and that of the putative macromolecule. By way of example, in some embodiments, such expected cleavage products may be compared with experimentally-observed cleavage products to confirm a degree to which a sequence of residues of a synthetic macromolecule corresponds to an expected sequence of the residues.

By way of example, in some embodiments, a plurality of experimentally-observed m/z ratios of a target macromolecule (e.g., an oligonucleotide) can be compared with a plurality of m/z ratios of the expected cleavage products of a putative macromolecule (e.g., an oligonucleotide) to determine a degree of correspondence between the sequence of the residues of the target macromolecule and that of the putative macromolecule. In some embodiments, the experimentally-observed m/z ratios of the cleavage products can be determined via MS/MS analysis of the target macromolecule.

The term “macromolecule” as used herein refers to a molecule having a plurality of residues, where each residue is coupled to at least one adjacent residue via at least one chemical bond. In some embodiments, the number of the residues of such a macromolecule can be, e.g., in a range of about 2 to about 5000. The term “a standard nucleotide” is used herein as the term nucleotide is conventionally employed to refer to an organic molecule composed of three subunit molecules: a base, a five-carbon sugar (ribose or deoxyribose), and a phosphate group. Nucleotides serve as monomeric units of the nucleic acid polymers deoxyribonucleic acid (DNA) and ribonucleic acid (RNA). The standard bases are guanine, adenine, cytosine, thymine, and uracil. The four bases in deoxyribonucleic acid (DNA) are guanine, adenine, cytosine, and thymine; in ribonucleic acid (RNA), uracil replaces thymine.

The term “a standard sugar” is used herein to refer to ribose and deoxyribose. The term “a standard peptide” refers to a macromolecule formed as a sequence of the 20 canonical amino acids.

The term “oligonucleotide” is used herein more broadly than the common use of this term in order to refer not only to a standard oligonucleotide having a plurality of standard structural units (e.g., standard bases such as adenine, cytosine, guanine, and thymine) that are connected to one another via a phosphodiester bond, i.e., a covalent bond formed between the 5′ phosphate group of one nucleotide and the 3′-OH group of another nucleotide, but also to molecules in which one or more of the standard structural units have been replaced with custom user-defined structural units. In embodiments, a user-defined oligonucleotide residue can be represented by five structural units. In some definitions of oligonucleotides in this broader context, the user defined structural units of a residue can have the same positional relationships relative to one another as the positional relationship that exist between structural units of a standard residue, e.g., a standard nucleotide.

For example, the term oligonucleotide, as used herein, can refer to a molecule in which one or more of the residues include a non-standard phosphate, e.g., one in which an oxygen atom of a standard phosphate group has been replaced with another atom or a chemical group.

The term “sequence” is used herein to indicate an ordered arrangement of a plurality of chemical moieties (e.g., nucleotides).

With reference to the flow chart of FIG. 1B, in one embodiment of a computer implemented method for determining expected cleavage products of a macromolecule, the macromolecule can be represented as a sequence of a plurality of residues bonded to one another via one or more chemical bonds, where each residue includes a core and a linker for coupling that residue to at least one adjacent residue. A linker of at least one of the residues can be defined as an assembly of two or more structural units, where the structural units are chemically bonded to one another via one or more chemical bonds. Further, in some embodiments, the core of at least one residue can be defined as an assembly of three or more structural units. In some embodiments, an entire residue, including its core and its linker, can be defined as an assembly of five structure units.

A digital data processor can be configured (e.g., programmed) to determine one or more expected intra- and inter-residue bond cleavages to determine the cleavage products of the macromolecule in response to application of energy thereto. For example, the digital data processor may be configured to determine expected bond cleavages between structural units of the core of the residue, between the structural units of the linker of the residue, as well as between linkers of adjacent residues, to determine the expected cleavage products of the macromolecule when the macromolecule undergoes cleavage. As the number of residues increases, so does the number of possible bond cleavages and the resulting cleavage products. The digital data processor can be configured to calculate these cleavage products by accounting for various possible permutations of the bond cleavages.

For example, the methods and systems according to the present teachings can be employed to determine the cleavage products of a macromolecule when that macromolecule undergoes cleavage in response to application of energy thereto. The applied energy can be generated via a plurality of different modalities. Some examples of such modalities include, without limitation, hydrolysis, electron capture dissociation, electron impact dissociation, collision-induced dissociation, depurination and depyrimidation.

In the following discussion, various features of embodiments of the present teachings are discussed primarily in connection with determining cleavage products of an oligonucleotide and/or a peptide, as those terms are used herein. However, the present teachings are not so limited, but can be applied to a variety of macromolecules, as discussed further below.

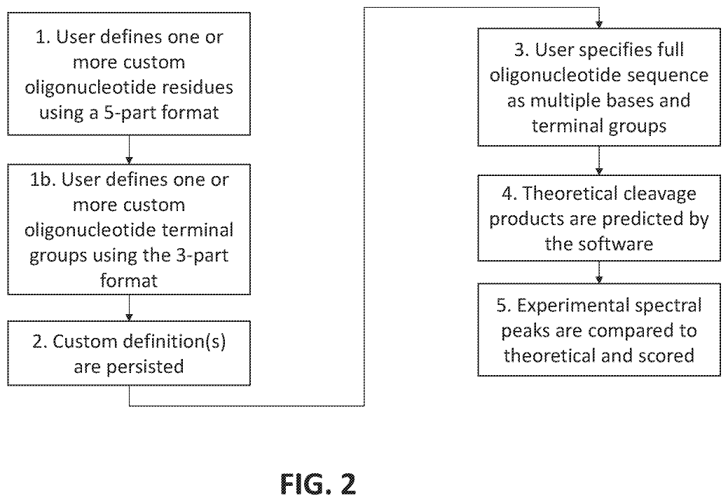

With reference to the flow chart of FIG. 2, in a method according to an embodiment of the present teachings, a user can define at least one residue of an oligonucleotide by specifying five structural units that collectively form such a residue. In some embodiments, the following five structural units can be employed for defining a residue: (1) a base (herein also referred to as a base moiety), a sugar (herein also referred to as a “sugar moiety”), a 3′ linker, a 5′ linker and a linking unit for linking adjacent residues via any of 5′ and 3′ linkers.

The structural units can be defined by the user so as to allow customization of the residue. In particular, in some embodiments, one or more user-defined structural units of a residue can be defined as a non-standard structural unit. For example, in some embodiments, the base moiety can be defined as having a structure other than those of standard nucleotide bases, namely, guanine, cytosine, uracil, adenine, thymine. In addition, or alternatively, in some embodiments, the sugar moiety can be a non-standard sugar (e.g., it can be a moiety other than a ribose or a deoxyribose). Similarly, the phosphate moiety can be a non-standard phosphate, e.g., a moiety in which one or more oxygens of a phosphate group have been replaced with another atom or a chemical group. Further, any of the 5′ and the 3′ linkers may be defined as any atom or a chemical group desired by the user. In some embodiments, any of the 5′ and the 3′ linker can be left empty (i.e., defined as being absent).

In some embodiments, an oligonucleotide may be defined as a sequence of residues, where one or more of those residues are user-defined non-standard residues. Alternatively, the oligonucleotide may be defined such that all of its residues are user-defined non-standard residues. In this manner, a variety of oligonucleotides with different molecular structures may be defined.

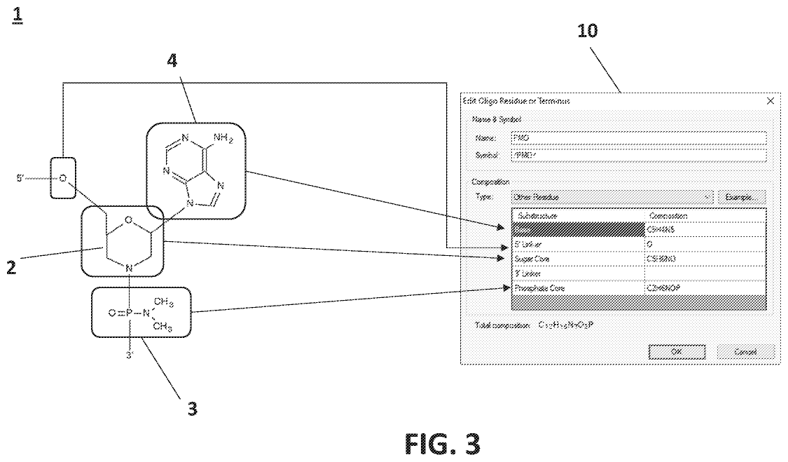

By way of illustration, FIG. 3 shows a user-defined oligonucleotide residue 1 in which both the sugar structural unit 2 as well as the phosphate structural unit 3 are defined as non-standard structural units. Specifically, in this custom definition of an oligonucleotide residue, the sugar structural unit 2 is a moiety different than a moiety that is commonly referred to as a sugar moiety. However, the position of this non-standard sugar moiety relative to other structural units of the residue is the same as that of standard ribose or a deoxyribose relative to other structural units of a standard nucleotide. Further, the phosphate structural unit 3 includes a phosphorus atom, which forms chemical bonds with an oxygen atom and an N(CH₃)₂ group. As such, it is different than a conventional phosphate in that one of the oxygen atoms of a conventional phosphate group has been replaced with the N(CH₃)₂ group and another oxygen atom (which would otherwise form a 3′ linker) is missing.

In contrast, in this user-defined residue, the base structural unit 4 is a standard base, i.e., adenine, and the 5′ linker is an oxygen atom.

With continued reference to FIG. 3, a user can employ a user interface element 10, which provides a plurality of data-entry windows to define the five structural units of a nucleotide of interest. In this case, for each of the structural units (which are referred to as “Substructure” in the user interface element), the user can identify the chemical composition of that structural unit. In this illustrative user interface element, the sugar structural unit is referred to as “Sugar Core” and the phosphate structural unit is referred to as the “Phosphate Core.” As noted above, in this example, the user has not entered any information regarding the 3′ linker, thereby indicating that the 3′ linker is missing.

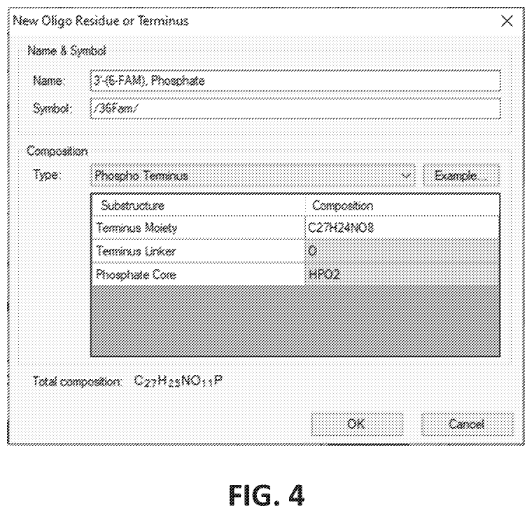

Referring again to the flow chart of FIG. 2 as well as FIG. 4, the user can optionally define one or more custom oligonucleotide terminal groups. In this example, the user can define, via an interface user element 10, a 5′ and/or a 3′ terminal group by identifying three structural units that collectively form that terminal group. In this example, the user interface element 10 identifies the three structural units as a “Terminus Moiety,” a “Terminus Linker,” and a “Phosphate Core.” In this example, the user has indicated that the Terminus Linker is oxygen and that the total composition of the terminal group is: C₂₇H₂₅NO₁₁P.

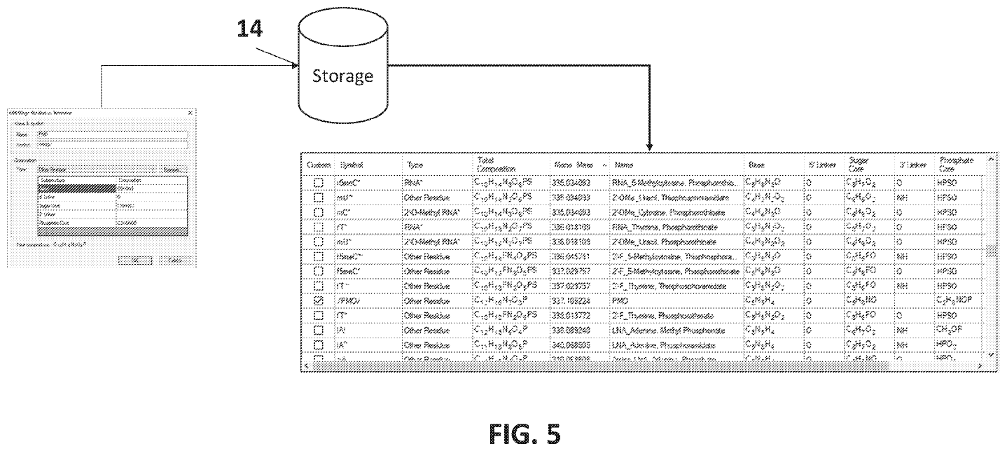

With continued reference to the flow chart of FIG. 2 as well as FIG. 5, the user-generated definition of the residue (and the terminal groups when such groups are defined) of the oligonucleotide can be stored in a persistent memory module, e.g., a database 14. By way of example, the storage of the user-defined residue based on the structural units defined by the user can be achieved using a variety of database structures known in the art, as informed by the present teachings. By way of example, and without limitation, FIG. 5 shows a database 14 that can store definitions (and other information) regarding one or more defined residues of one or more oligonucleotides, e.g., in a tabular form.

In this example of a tabular compilation of the information regarding each residue, in addition to the five user-defined structural units, an identifying symbol, the type, the total composition, the molecular mass, and the chemical name of the residue are also included.

In some embodiments, the database can be configured to compare a user-defined residue with those previously stored in the database. If the user-defined residue is new (i.e., it is not already stored in the database), the database can store the user-defined information, e.g., in a manner discussed above.

Referring again to the flow chart of FIG. 2, once the residues are defined, and optionally stored in a database if new, the user can define an oligonucleotide as a sequence of standard and/or user-defined residues, including any terminal groups.

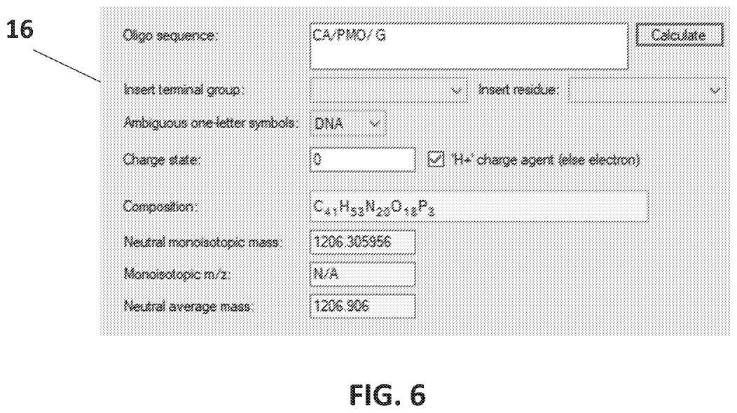

By way of illustration, FIG. 6 shows a user interface element 16 that can be employed by a user to define the oligonucleotide. In particular, in this example, the user can enter via a window of the user interface element 16 labeled as “Oligo sequence,” an ordered sequence of residues that collectively form the oligonucleotide of interest.

In some embodiments, a user-defined residue can be assigned a unique symbol, e.g., a symbol comprising one or more alphanumeric characters. For example, in some such embodiments, a unique alphanumeric character may be assigned to each structural unit of a residue to provide a symbolic representation of that residue. In some embodiments, the standard residues (e.g., standard bases and/or sugar moieties) may be represented by conventional symbols employed for their representation.

In this example, the oligonucleotide is defined as an ordered sequence of four residues, which include a standard cytosine (C) residue, a standard adenine (A) residue, a user-defined residue (PMO) (See, e.g., FIG. 6), and a standard guanine (G) residue.

In this example, in response to the entry of the information regarding the residues, the user interface element 16 provides the chemical composition, and the molecular weight of the resulting intact oligonucleotide molecule. Although in this example the oligonucleotide includes only four residues, the present teachings are not limited to any particular number of oligonucleotide residues. In fact, the present teachings can be employed to define an oligonucleotide having any number of residues. By way of example, the number of the residues can be in a range of 2 to about 1000, e.g., in a range of 50 to 500. In some embodiments, the definition of the oligonucleotide generated in a manner discussed above can be employed to determine, using a digital data processing unit, the expected cleavage products of the oligonucleotide when such an oligonucleotide undergoes cleavage, e.g., as a result of application of energy thereto, e.g., via a chemical reaction, particle impact and/or radiation.

In some embodiments, the cleavage of the oligonucleotide occurs as a result of a fragmentation process employed in an MS/MS analysis of the oligonucleotide.

In some embodiments, the cleavage products can be identified based on expected cleavage of one or more bonds, either individually or in combination, when the oligonucleotide is subjected to energy from an energy source.

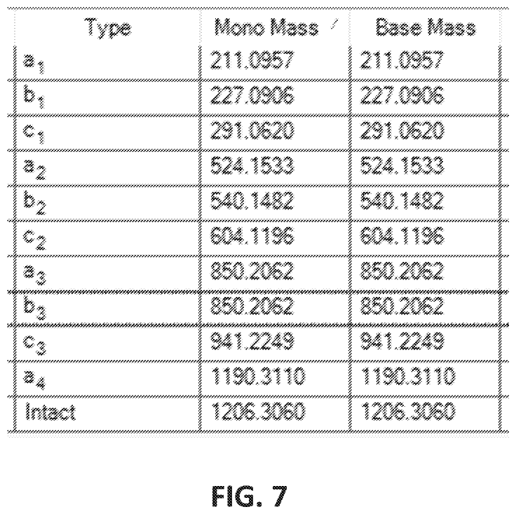

By way of illustration, FIG. 7 presents the neutral cleavage products ‘a’, ‘b’, and ‘c’, which are generated for the oligonucleotide discussed above (which is represented by the following sequence of symbols: “C A/PMO/G.”) when that oligonucleotide undergoes cleavage.

The ‘a’ and ‘b’ cleavage products typically differ by one oxygen atom (mass of approximately 16 Da). In contrast, in this example, the a₃ and b₃ cleavage products are identical since the PMO residue has no 3′ linker. Further, the ‘b’ and ‘c’ cleavage products typically differ by PO₂H (mass of approximately 64 Da), however, the b₃ and c₃ differ by C₂H₆NOP (i.e., the non-standard “Phosphate Core”).

Referring again to the flow chart of FIG. 2, in some embodiments, the experimentally-observed cleavage products of a target oligonucleotide can be compared with the expected cleavage products of a putative oligonucleotide to determine the degree of correspondence of the molecular structure of the target oligonucleotide with that of the putative oligonucleotide.

By way of example, the mass spectral peaks observed in an experimental MS/MS spectrum of a target oligonucleotide can be compared with expected mass spectral peaks associated with the expected fragments of the defined oligonucleotide to determine the degree of correspondence between the molecular structure of the target oligonucleotide and that of the defined oligonucleotide.

In some embodiments, such comparison between the experimentally-observed mass peaks and the m/z ratios associated with mass peaks corresponding to the expected fragments is performed within a predefined m/z tolerance. For example, an experimentally-observed mass peak can be identified as corresponding to an expected mass peak when the difference between the m/z ratios of the two mass peaks is less than, e.g., about 0.5 Da for a low-resolution quadrupole mass spectrometer or about 0.05 Da or lower for a higher-resolution time-of-flight (TOF) mass spectrometer.

In some embodiments, the degree of the correspondence between the molecular structure of a target macromolecule (e.g., a target oligonucleotide) with that of a putative macromolecule (e.g., a putative oligonucleotide) can be quantified using a scoring system, such as that discussed above.

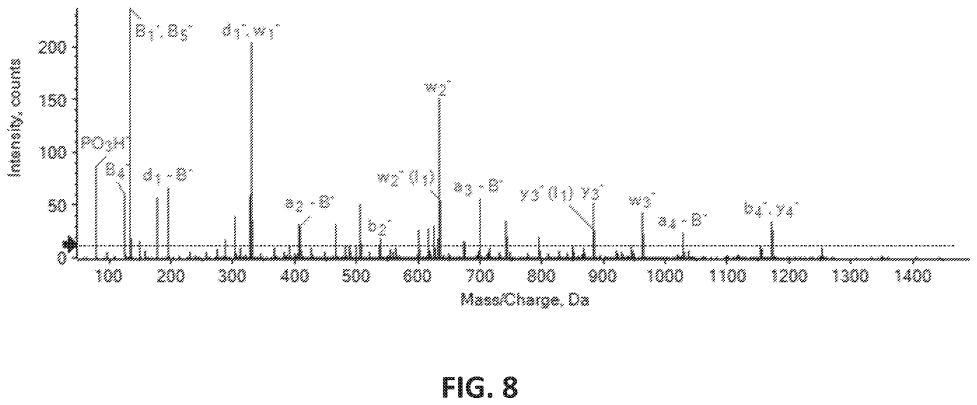

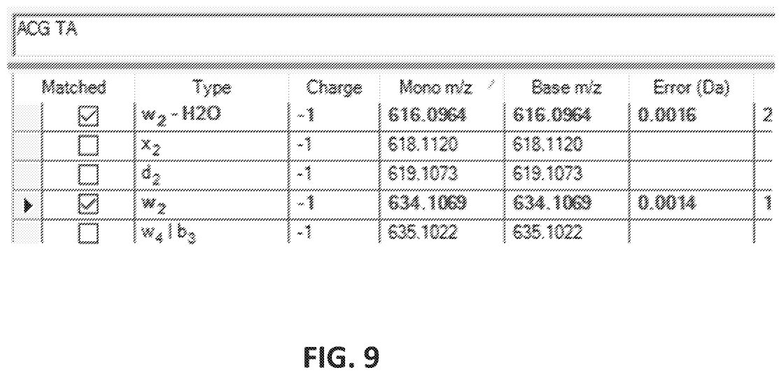

By way of illustration, FIGS. 8 and 9 show, respectively, an experimentally-observed MS/MS spectrum of a five-residue nucleotide and a portion of a table listing mass-to-charge ratios for the expected fragments. The labeled mass peaks are those that match the respective expected mass peaks within a predefined m/z tolerance of 20 ppm (parts per million), and the other mass peaks are either not matched to an expected mass peak or their signal levels are below a defined threshold shown by the dotted line.

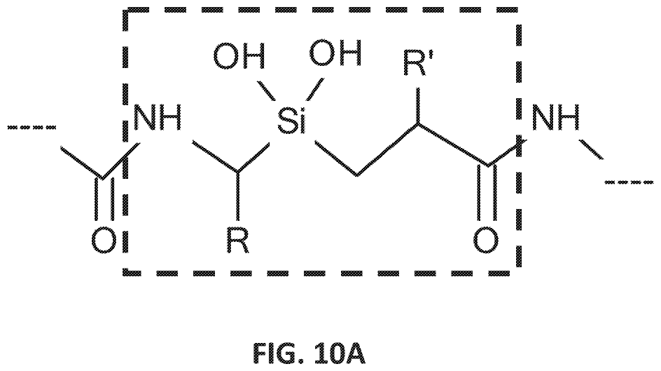

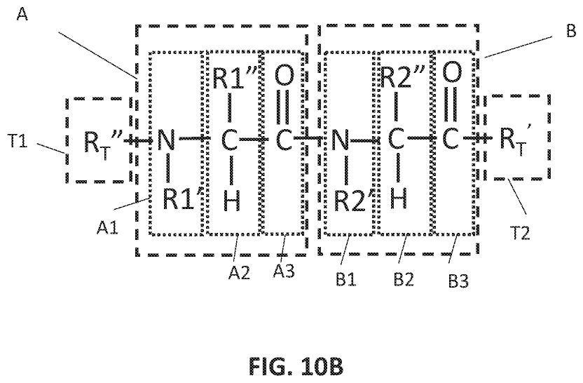

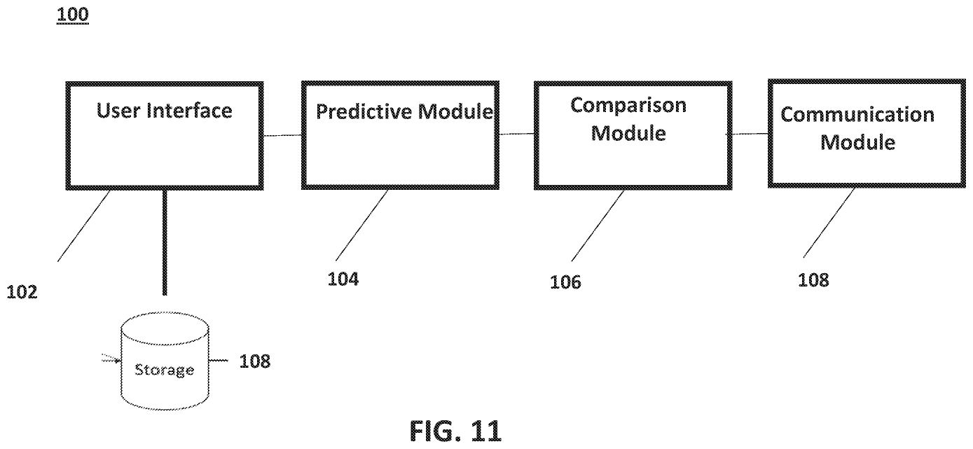

FIG. 10 schematically depicts an example of an embodiment of a system 100 for implementing methods according to the present teachings. The system 100 includes a user interface 102 that provides a plurality of user interface elements, such as those discussed above in connection with FIGS. 3-6, to allow a user to define one or more user-defined custom residues of a macromolecule. The user interface can further allow the user to assign a unique symbol (e.g., an alphanumeric symbol) to the defined residue.

In this embodiment, the system 100 further includes a database (e.g., a relational database) 104 in communication with the user interface for storing the user-defined residues. As discussed above, in some embodiments, the database can determine whether an inputted user-defined residue is new, and if so, it will store that residue. When storing the information regarding the residue, the database can associate the user-defined residue with its respective symbol to allow a user to identify the residue via that symbol for retrieving the residue for defining an oligonucleotide.

The user interface 102 can further include a user interface element, such as the user interface element 16 discussed above, that allows a user to define a macromolecule of interest via the identification of a sequence of the residues of that macromolecule.

With continued reference to FIG. 10, the system 100 further includes a predictive module 104 that is in communication with the user interface 102 to receive information regarding the sequence of the residues of a defined macromolecule.

The predictive module 104 is configured to determine the cleavage products generated when the macromolecule is subjected to an energy from an energy source. For example, the predictive module 104 can be configured to identify one or more chemical bonds between various structural units of each residue and chemical bonds between the different residues that are known to be susceptible to breakage in response to the application of the identified energy.

The predictive module 104 can utilize a digital processor to compute cleavage products that are expected to result from the breakage of such bonds, individually and in various combinations. For example, the predictive module 104 can compute various permutations of expected bond breakages to arrive at the expected cleavage products. By way of example, the predictive module 104 can use available information regarding bond breakage via various mechanisms employed in MS/MS analysis of oligonucleotides, e.g., collision-induced dissociation, electron capture dissociation, electron impact dissociation, to determine the cleavage products of a user-defined oligonucleotide when subjected to such fragmentation.

As discussed above, in some embodiments, the structural units of the residues are defined such that the breakage of bonds between those structural units and between adjacent residues will generate the most significant cleavage products.

In some embodiments, the system 100 can further include a comparison module 106 that can receive information regarding the expected cleavage products when the macromolecule is subjected to the identified energy modality as well as information regarding experimentally-observed cleavage products generated by exposing a target macromolecule to the identified energy modality so as to determine the degree of correspondence between the molecular structure of the target macromolecule and that of the putative macromolecule.

By way of example, in some embodiments in which an experimentally-observed MS/MS spectrum of a target oligonucleotide is compared with an expected MS/MS spectrum of a putative oligonucleotide, a mass peak in one spectrum can be considered as corresponding to a mass peak in the other spectrum when a difference between the m/z ratios associated with those mass peaks is less than a predefined tolerance, e.g., as defined above

The system 100 can include a communications module 108 that can allow the system to communicate with external devices, e.g., external databases as well as instruments that are employed to generate the experimentally-observed cleavage products, e.g., a mass spectrometer, to receive and send data to such devices.

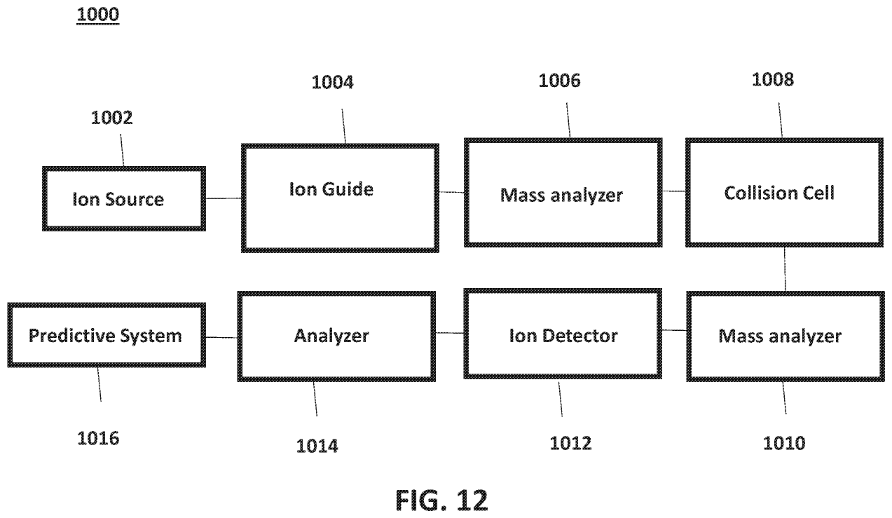

In some embodiments, a predictive system according to the present teachings can be incorporated in a mass spectrometer. By way of example, FIG. 12 schematically depicts a mass spectrometry system 1000 according to an embodiment, which allows comparing a degree of correspondence between a molecular structure of a target macromolecule with that of a putative macromolecule, via a comparison of MS/MS mass peaks expected for the cleavage products of the putative macromolecule with mass peaks observed via MS/MS analysis of the target molecule.

In this embodiment, the mass spectrometry system 1000 includes a predictive system 1016 according to an embodiment of the present teachings, such as the above system 100, that can receive information regarding the sequence of the residues of an oligonucleotide defined by a user and is configured to determine the cleavage products that can be generated if the defined oligonucleotide were to be subjected to a particular MS/MS analysis, e.g., an MS/MS analysis in which the fragmentation of the precursor ions is achieved via electron capture dissociation or electron impact dissociation.

The mass spectrometry system 1000 can further include an ion source 1002 for receiving a sample of a target oligonucleotide and ionize at least a portion of that sample so as to generate a plurality of precursor ions. The precursor ions can be received by an ion guide 1004, via passage through openings provided in a curtain plate and an orifice plate (not shown), where the ion guide can provide focusing of the ions to generate an ion beam. In some embodiments, the ion guide 1004 can be implemented using a plurality of rods that are arranged relative to one another in a multipole configuration (e.g., a quadrupole configuration) and to which RF and/or DC voltages can be applied, e.g., to provide radial confinement of ions.

A first mass analyzer 1006 positioned downstream of the ion guide 1004 can receive the ions and can select those ions having a desired m/z ratio. Similar to the ion guide 1000, the first mass analyzer 1006 can be implemented using a plurality of rods arranged in a multipole configuration to which RF and DC voltages can be applied. By way of example, the application of a resolving DC voltage across at least two of the rods can allow the selection of ions having m/z ratios of interest by ensuring that those ions will follow stable trajectories through the ion guide while ions with different m/z ratios will experience unstable trajectories and thus prevented from exiting the mass filter.

In this embodiment, the ions passing through the first mass filter will enter a collision cell 1008 in which at least a portion of the ions will undergo fragmentation so as to generate a plurality of product ions. The product ions can be received by a second mass analyzer 1010, which can select product ions having a desired m/z ratio for detection by a downstream ion detector 1012. The m/z selection window of the second mass analyzer can be swept to detect the product ions with different m/z ratios generated as a result of fragmentation of a selected precursor ion. In response to the detection of the product ions, the ion detector generates a plurality of ion detection signals that are received by an analysis module 1014, which processes those signals to generate a mass spectrum of the product ions.

In this embodiment, the analysis module 1014 is in communication with the comparison module of the system 1016 to send the generated mass spectrum thereto. In some embodiments, such communication is a wired communication while in other embodiments, it can be a wireless communication. As discussed above, the comparison module can compare a plurality of experimentally-observed mass peaks to a plurality of expected mass peaks to determine the degree of correspondence between them.

As noted above, the present teachings are not limited to defining oligonucleotides, but can also be employed for defining other types of macromolecules. An example of such a macromolecule is a peptide. By way of illustration, FIG. 10A shows a user-defined residue of such a peptide, which includes carbon-carbon and carbon-nitrogen backbone bonds as well as terminal amino and carbonyl groups.

However, unlike standard amino acids, it includes a silicon atom in its backbone, which forms silicon-carbon bonds, and is also coupled to two hydroxyl groups. Moreover, it includes pendant groups R and R′ other than hydrogen, which can be a variety of different chemical groups.

By way of further illustration, FIG. 10B shows two residues (i.e., residues A and B) and two terminal groups T1 and T2 of another user-defined peptide as that term is used herein. Both residues include carbon-carbon and carbon-nitrogen backbone bonds, and a C═O moiety. Both residues A and B further include a number of pendant groups, which can be a variety of different chemical groups. While in some cases the pendant groups can be selected such that residue A, B, or both would correspond to a canonical amino acid, in other cases, the pendant groups of residue A, B, or both can be selected such that the resultant peptide would be a non-standard peptide.

Residue B, in turn, includes carbon-carbon and carbon-nitrogen backbone bonds, and a terminal carbonyl group. But rather than having an NH group, as in a standard peptide residue, it includes an NRO group.

With continued reference to FIG. 10B, residue A can be defined as having two linker structural units A1 and A2, and a core structural unit A3. Similarly, residue B can be defined as having two linker structural units B1 and B2, and a core structural unit B3. By considering various expected bond cleavages within the linker and the core structural units of each residue as well as a bond cleavage between the two residues, a method according to an embodiment of the present teachings can predict expected cleavage products for the peptides A and B.

As discussed above, in some embodiments, the experimentally-observed cleavage products of a target peptide can be compared with the expected cleavage products of a putative peptide to arrive at a measure of correspondence between the molecular structure of the putative peptide and that of the target peptide, which is expected to have the same molecular structure as that of the putative peptide.

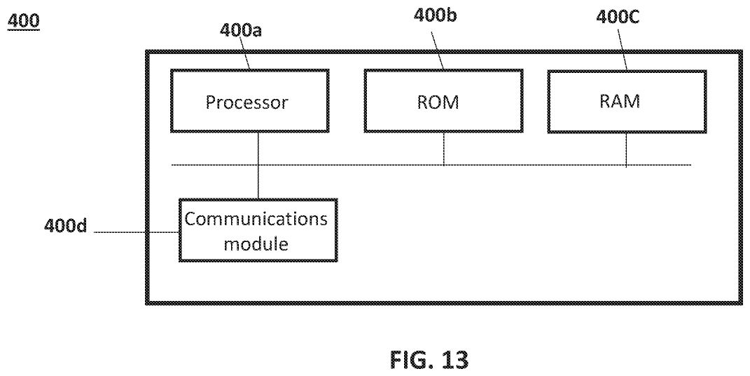

A system according to the present teachings, such as the above system 100, can be implemented in hardware, software and/or firmware in a manner known in the art as informed by the present teachings. By way of example, FIG. 13 schematically depicts an example of an implementation of such a system 400, which includes a processor 400a (e.g., a microprocessor), at least one permanent memory module 400b (e.g., ROM), at least one transient memory module (e.g., RAM) 400c, and a bus 400d, among other elements generally known in the art. The bus 400d allows communication between the processor and various other components of the system. In this example, the system 400 can further include a communications module 400e that is configured to allow sending and receiving signals, e.g., for communicating with various databases.

Instructions for operating various components of system 400, e.g., for determining expected bond cleavages and the expected cleavage products, comparing expected cleavage products with experimentally-observed cleavage products, and/or operating the user interface can be stored in the permanent memory module 400b and can be transferred into the transient memory module 400c during runtime for execution. As noted above, although the predictive module and the comparison module are shown as separate modules, in some embodiments, their functionalities can be integrated within the same module.

Although some aspects have been described in the context of a system and/or an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus. Some or all of the method steps may be executed by (or using) a hardware apparatus, like for example, a processor, a microprocessor, a programmable computer or an electronic circuit. In some embodiments, one or more of the most important method steps may be executed by such an apparatus.

Depending on certain implementation requirements, embodiments of the invention can be implemented in hardware and/or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

It should be understood that the methods and systems according to the present teachings can be employed for predicting the cleavage products of a variety of macromolecules.

What is claimed is:

1. A computer implemented method for determining expected cleavage products of at least one macromolecule having a plurality of residues bonded to one another, comprising:
defining at least one of said residues as having a core and at least one linker, wherein said at least one linker is defined as an assembly of two or more structural units that are coupled to one another via one or more chemical bonds,
representing said macromolecule as a sequence of said residues such that each residue is bonded to an adjacent residue via at least one chemical bond, and
using a digital data processor to determine one or more expected bond cleavages, if any, between the structural units of said at least one linker and between adjacent residues when said macromolecule undergoes cleavage so as to determine expected cleavage products of said macromolecule.

2. The computer implemented method of claim 1, wherein said macromolecule undergoes cleavage in response to application of energy thereto.

3. The computer implemented method of claim 2, wherein said energy is generated via any of a chemical reaction, particle impact and radiation generated by a radiation source.

4. The computer implemented method of claim 1, wherein at least one of said linker structural units comprises a plurality of atoms that are bonded to one another via one or more chemical bonds.

5. The computer implemented method of claim 4, wherein said digital data processor is configured to determine expected bond cleavages, either individually or in combination, of said chemical bonds formed between said plurality of atoms of said at least one of said structural units for determining said expected cleavage products.

6. The computer implemented method of claim 1, wherein said core of said at least one of said residues is represented as a sequence of at least two structural units forming chemical bonds with one another.

7. The computer implemented method of claim 6, wherein said digital data processor is further configured to determine one or more expected core cleavages of the chemical bonds between said core structural units, if any, and to use said expected core cleavages, in addition to said expected linker bond cleavages, if any, to determine said expected cleavage products of said macromolecule.

8. The computer implemented method of claim 1, wherein said macromolecule comprises an oligonucleotide.

9. The computer implemented method of claim 6, wherein said core structural units comprise a base and a sugar.

10. The computer implemented method of claim 9, wherein said base comprises a non-standard base.

11. The computer implemented method of claim 1, wherein said linker structural units comprise a phosphorus-containing group, a 5′ linker atom and a 3′ linker atom, and wherein optionally at least one of the structural units of said linker comprises silicon, and wherein optionally said silicon-containing structural unit comprises Si(OH)₂.

12. The computer implemented method of claim 1, wherein at least one of said residues comprises any of an amino, a hydroxy-amino and a carbonyl group.

13. The computer implemented method of claim 2, wherein said energy is provided by any of hydrolysis, electron capture dissociation, electron impact dissociation, collision-induced dissociation, depurination and depyrimidation.

14. The computer implemented method of claim 1, further comprising assigning a unique symbol to each of the structural units of said at least one of said residues so as to generate a symbolic representation of said at least one of said residues.

15. The computer implemented method of claim 1, further comprising comparing said determined cleavage products with experimentally-observed cleavage products of a target macromolecule to determine a degree of correspondence between said macromolecule and the target macromolecule.

16. The computer implemented method of claim 1, further comprising storing the definition of the macromolecule in a database.

17. A mass spectrometry system, comprising
a predictive system comprising at least one digital data processor configured to determine cleavage products of a putative macromolecule, which comprises a plurality of residues coupled to one another by one or more chemical bonds and in which at least one of said residues is defined as having a core and at least one linker, wherein said at least one linker is defined as an assembly of two or more structural units,
an ion source for receiving a target macromolecule and ionizing said target macromolecule so as to generate a plurality of precursor ions,
at least one ion guide for receiving said precursor ions and providing focusing thereof,
a first mass analyzer positioned downstream of said at least one ion guide for selecting precursor ions having a target m/z ratio,
a collision cell positioned downstream of said mass analyzer for causing fragmentation of at least a portion of said selected precursor ions into a plurality of product ions,
a second mass analyzer for selecting at least a portion of the product ions having a target m/z ratio,
a detector for detecting said selected product ions and generating one or more detection signals in response to the detection of said product ions, and
an analysis module configured to receive said detection signals and to generate an experimentally-observed MS/MS spectrum of said target macromolecule,
wherein said analysis module is configured to transmit said experimentally-observed MS/MS spectrum to said predictive system and said predictive system is configured to compare said experimentally-observed MS/MS spectrum with a spectrum corresponding to said theoretically determined cleavage products so as to determine a degree of correspondence between said putative macromolecule and said target macromolecule.

18. The mass spectrometry system of claim 17, wherein said predictive system further comprises:
a user interface operating under control of said microprocessor, said user interface comprising at least one user interface element configured to allow a user to identify at least one residue of said macromolecule as a sequence of a plurality of structural units chemically bonded to one another, said at least one user interface element further configured to allow the user to define said macromolecule as a sequence of chemically-bonded residues comprising said at least one residue and one or more additional residues, and
a predictive module operating under control of said microprocessor and being in communication with said user interface for receiving information regarding said residues, said predictive module being configured to determine theoretically one or more expected bond cleavages of said macromolecule when said macromolecule undergoes cleavage so as to predict expected cleavage products of said macromolecule.

19. The mass spectrometry system of claim 18, wherein said predictive module comprises a comparison module configured to compare the experimentally-observed MS/MS spectrum with a theoretical mass spectrum corresponding to said expected cleavage products so as to determine said degree of correspondence between the target macromolecule and said putative macromolecule.

20. The mass spectrometry system of claim 18, wherein said comparison module identifies an association between a mass peak in the experimentally-observed MS/MS spectrum and a mass peak in said expected mass spectrum when a difference between m/z ratios of said mass peaks is less than a predefined threshold.
